(19) United States
(12) Patent Application Publication
(10) Pub. No.: US 2023/0346212 A1
(43) Pub. Date: Nov. 2, 2023

(54) Internal Cleaning of Laparoscopic Lens With Wiper

(71) Applicant: Matthew D. Dovgan, Phoenix, AZ (US)

(72) Inventor: Matthew D. Dovgan, Phoenix, AZ (US)

(21) Appl. No.: 17/861,333

(22) Filed: Jul. 11, 2022

Related U.S. Application Data
(60) Provisional application No. 63/337,049, filed on Apr. 29, 2022.

(51) Int. Cl. A61B 1/313; A61B 1/12; A61B 1/06

(52) U.S. Cl. CPC A61B 1/3132; A61B 1/0676; A61B 1/126; A61B 1/127; A61B 2017/00309

(57) ABSTRACT

A medical scope trocar having a wiper therein configured for cleaning of a medical scope lens located within the trocar, and a method for the cleaning of a medical scope lens without removing it from a trocar, by locating a wiper within the trocar and causing relative movement between the wiper and the lens.

### FIELD

The present disclosure relates to the cleaning of lenses used in laparoscopic medical procedures. More particularly, the disclosure relates to trocar assemblies having a wiper located therein configured for cleaning of a laparoscopic lens located within the trocar, and to methods for the cleaning of a laparoscopic lens without removing it from a trocar.

### BACKGROUND

Improvement is desired in the provision of medical scopes, such as endoscopes, laparoscopes, and the like trocar assemblies having a lens and utilized in laparoscopic medical procedures. Laparoscopic and other medical scope lenses such as camera lenses get dirty or fogged during procedures and need to be cleaned periodically during a procedure. Conventionally this is done by removing the lens from the trocar. This is inconvenient and time consuming. What is desired is a way to clean the lens without having to remove it from the trocar.

This would help to minimize the risk of contamination, decrease the risk for postoperative infections, and increase the efficiency of the surgical procedure by cutting down the time it takes to fully remove and clean the lens. The devices and methods would serve the needs of patients who need a laparoscopic procedure, and be able to be used in robotic assisted procedures as well. This may also decrease the amount of time the patient is under anesthesia.

### SUMMARY

The present disclosure advantageously provides trocar assemblies having a wiper therein configured for cleaning of a laparoscopic lens located within the trocar. The disclosure also provides methods for the cleaning of a medical scope lens without removing it from a trocar, by locating a wiper within the trocar.

In one aspect, a medical scope according to the disclosure includes a trocar; an illuminated lens tube slidably disposed within the trocar to be extendable out of the trocar and retractable into a distal end of the trocar, the lens tube including a distal tip; and a wiper disposed internally within the trocar and configured for passage of the lens tube through the wiper, wherein relative movement between the lens tube and the wiper cleans the distal tip of the lens tube.

In another aspect, a method for the cleaning of a lens of a medical scope includes the steps of providing a trocar; providing an illuminated lens tube slidably disposed within the trocar to be extendable out of the trocar and retractable into a distal end of the trocar, the lens tube including a distal tip; providing a wiper disposed internally within the trocar and configured for passage of the lens tube through the wiper; and performing relative movement between the lens tube and the wiper to clean the distal tip of the lens tube.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1A depicts a laparoscope having an internal wiper according to the disclosure.

FIG. 1B is an enlarged view of a portion of FIG. 1A.

FIG. 2 is an end view of the laparoscope.

FIG. 3 is a perspective view of the wiper component.

FIGS. 4-6 are cross-sectional views depicting use of the wiper to clean a lens of the laparoscope.

FIGS. 7-9 are cross-sectional views depicting use of the wiper to clean a lens of the laparoscope.

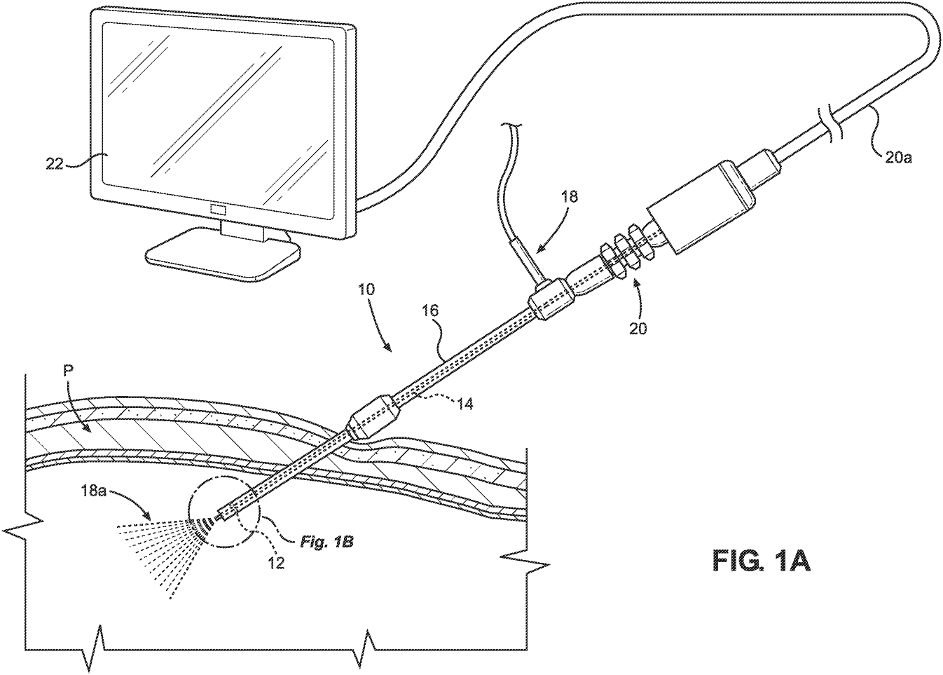

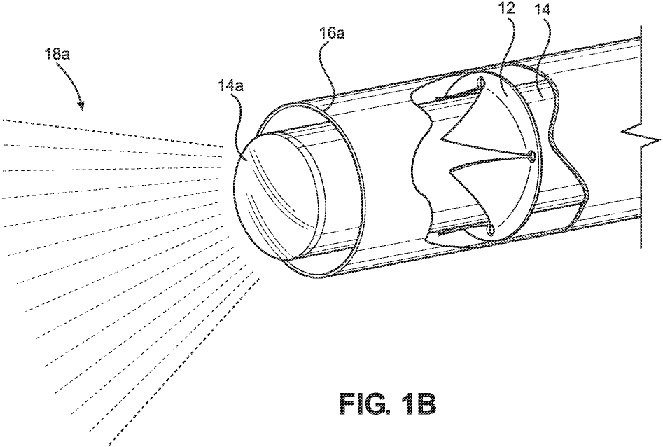

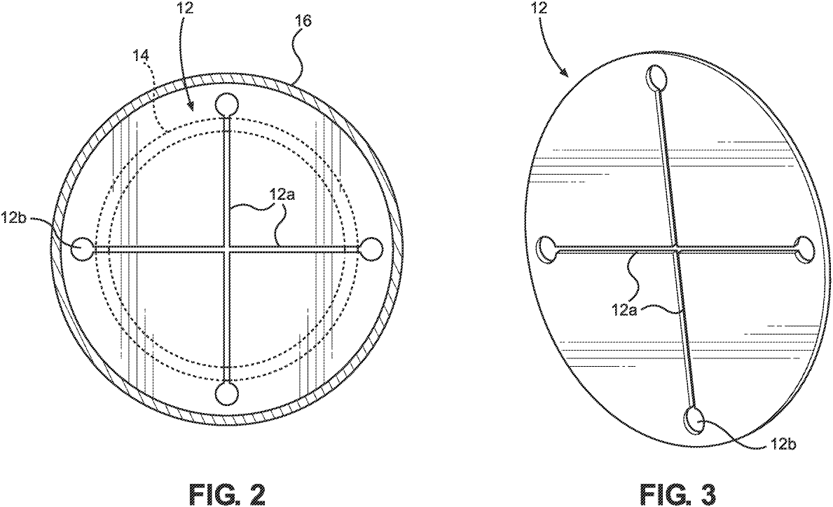

### DETAILED DESCRIPTION

With reference to the drawings, the disclosure provides a medical scope depicted as a laparoscope 10 configured to have an internal wiper 12 according to the disclosure. The wiper 12 is configured for cleaning a distal lens tip 14a of a lens tube 14 of the laparoscope without removal of the lens tip 14a from the laparoscope 10.

The laparoscope 10 functions to enable the lens tip 14a to be deployed internally within a patient P through an incision. The laparoscope 10 may be any conventional laparoscope. The lens tube 14 may be any type of lens structure commonly used for laparoscopes. Commonly utilized lens structures include fiber optics, light guided rods, and the like. It will be appreciated that the wiper of the disclosure may be applied to any medical scope device of the type having an illuminated lens tube inserted through a trocar, such as an arthroscope.

As shown, the laparoscope 10 is an electronic laparoscope having an outer cannula or trocar 16 within which the lens tube 14 is slidably or movably disposed for linear movement to be extendable out of and retractable into a distal end 16a of the trocar 16. The lens tube 14 is also rotatable within the trocar 16. A light source 18, typically a light source provided by light emitting diodes, is optically connected to the lens tip 14a to emit illumination 18a from the lens tip 14a. In the depicted electronic laparoscope 10, optical images from the lens tube 14 are converted by a camera in the form of an electronic processor 20 to electronic signals that are transmitted via a cable 20a to a video monitor 22 for display of the images for viewing by the surgeon.

The laparoscope 10 may be a non-electronic laparoscope having an image viewer on the proximal end for direct viewing instead of connected to a monitor. In any event, in relevance to the present disclosure, the lens tube 14 is slidably disposed in a cannula or trocar 16 and the wiper 12 is located internally within the cannula or trocar 16 so that relative movement of the lens tip 14a and the wiper 12 may be accomplished for cleaning of the lens tip 14a.

The wiper 12 is located internally within the trocar 16 and is configured for cleaning the lens tube 14 and the lens tip 14a of the lens tube 14 while the lens tip 14a is located within the trocar 16 to avoid the need for removal of the lens tube 14 from the trocar 16. The wiper 12 is of one-piece unitary construction and made of a flexible medical grade polymeric material suitable for the surgical environment. The wiper 12 is fixedly positioned at a location within the trocar 16. This location may desirably be proximate to the tip 16a such as about 1-6 inches from the tip 16a but may be located at other locations therein. The wiper 12 provides a cleaning structure that contacts the lens tip 14a to wipe and otherwise dispel surgical debris or fog or the like matter that may accumulate on the lens tip14a during a surgical procedure and obscure the view.

The wiper 12 as depicted is disk-shaped as the depicted trocar 16 is circular. In this regard, the wiper 12 is configured as a generally flat shape corresponding to the cross-section of the interior of the trocar 16. However, the wiper 12 may be thicker or deeper to provide more surface area for cleaning of the lens tip 14a.

The wiper 12 includes slits 12a desirably formed in the shape of a cross. The slits 12a move or flex or deform to enable the lens tube 14 to pass through the slits 12a. The ends 12b of the slits 12a may be provided as enlarged curved portions to facilitate flexing of the material of the wiper 12 bounding the slits 12a and to avoid cracking of the material.

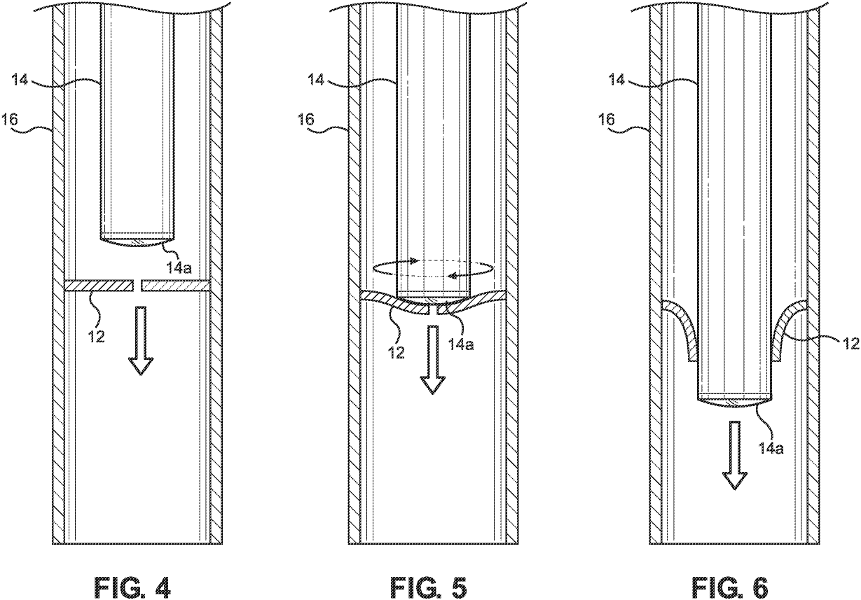

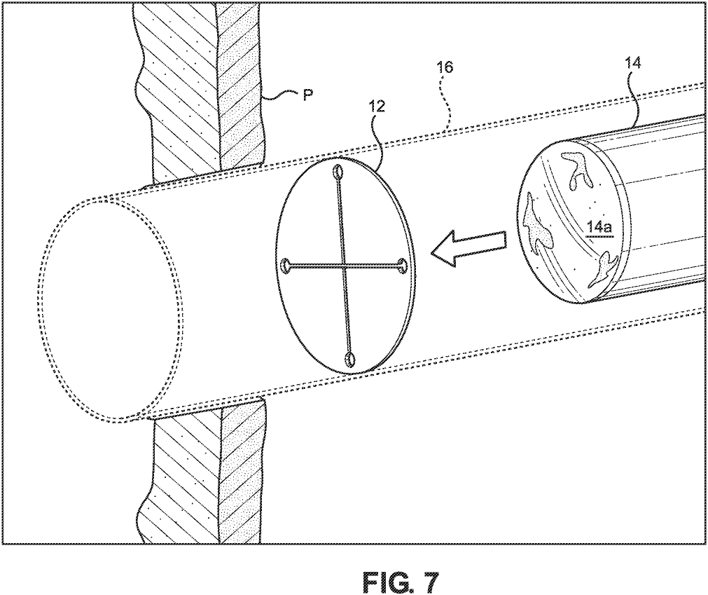

As depicted in FIGS. 4-6 and 7-9, in use the lens tube 14 is movable along the length of the trocar 16 and rotatable therein as shown by the respective arrows, and the wiper 12 flexes along the slits 12. These actions provide relative motion between the lens tube 14 and the wiper 12 to effect contact between the lens tube 14 and the lens tip 14a to mechanically dispel surgical debris (e.g., tissue, blood, fat and the like) or fog or the like from the lens tube 14 and most importantly the lens tip 14a to effect cleaning thereon. The surgical debris/fog is depicted by the shading on the lens tip 14a in FIG. 7. This cleaning of the lens tip 14a may be required repeatedly during a laparoscopic procedure and can be performed as many times as needed.

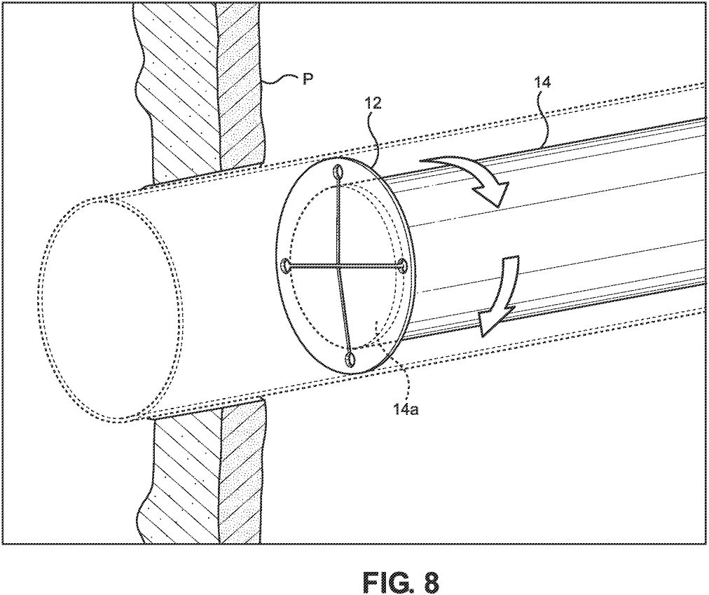

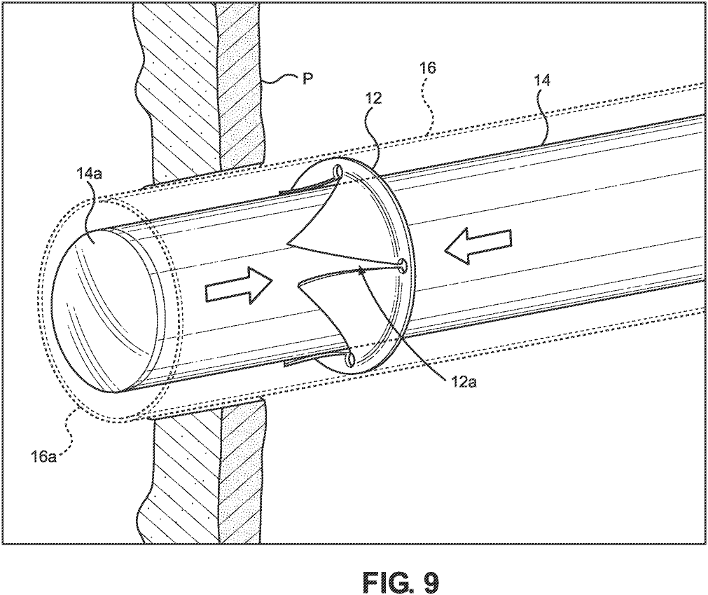

For example, as shown in FIGS. 4 and 7, the lens tube 14 is first retracted past the wiper 12. Next, as shown in FIGS. 5 and 8, a twisting motion is imparted to the lens tube 14 to dispel the debris. Then the surgical procedure can advance (FIGS. 6 and 9) without having to remove the lens tip 14a from the trocar 16.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated.

1. An illuminated medical scope, comprising:
a trocar;
an illuminated lens tube slidably disposed within the trocar to be extendable out of the trocar and retractable into a distal end of the trocar, the lens tube including a distal tip; and
a wiper disposed internally within the trocar and configured for passage of the lens tube through the wiper, wherein relative movement between the lens tube and the wiper cleans the distal tip of the lens tube.

2. The scope of claim 1, wherein relative movement between the lens tube and the wiper comprises movement of the lens tube relative to the wiper.

3. The scope of claim 2, wherein relative movement comprises rotational movement of the lens tube relative to the wiper and/or linear movement of the lens tube relative to the wiper.

4. The scope of claim 1, wherein the wiper comprises a disk with slits therein.

5. The scope of claim 4, wherein the relative movement comprises flexing of the wiper.

6. The scope of claim 1, wherein the scope is a laparoscope.

7. A method for the cleaning of a lens of an illuminated medical scope, comprising the steps of:
providing a trocar;
providing an illuminated lens tube slidably disposed within the trocar to be extendable out of the trocar and retractable into a distal end of the trocar, the lens tube including a distal tip;
providing a wiper disposed internally within the trocar and configured for passage of the lens tube through the wiper; and
performing relative movement between the lens tube and the wiper to clean the distal tip of the lens tube.

8. The method of claim 7, wherein the step of providing relative movement between the lens tube and the wiper comprises providing movement of the lens tube relative to the wiper.

9. The method of claim 8, wherein providing movement of the lens tube relative to the wiper comprises providing rotational movement of the lens tube relative to the wiper and/or linear movement of the lens tube relative to the wiper.

10. The method of claim 7, wherein the wiper comprises a disk with slits therein.

11. The method of claim 10, wherein the providing relative movement of the lens tube relative to the wiper comprises flexing the wiper.
